(19) United States
(12) Patent Application Publication
(10) Pub. No.: US 2016/0030443 A1
(43) Pub. Date: Feb. 4, 2016

(54) METHODS AND COMPOSITIONS FOR MODULATING REGULATORY T CELL FUNCTION

(71) Applicant: Rongfu WANG, Houston, TX (US)

(72) Inventor: Rongfu WANG, Houston, TX (US)

(21) Appl. No.: 14/775,725

(22) PCT Filed: Mar. 14, 2014
(86) PCT No.: PCT/US14/26940

§ 371 Date: Sep. 14, 2015

Related U.S. Application Data
(60) Provisional application No. 61/781,024, filed on Mar. 14, 2013.

(51) Int. Cl. A61K 31/5383; A61K 31/47; A61K 31/55; A61K 39/39; A61K 31/4741; G01N 33/50; A61K 45/06; A61K 31/496; A61K 31/519

(52) U.S. Cl. CPC A61K 31/5383; A61K 31/496; A61K 31/47; A61K 31/55; A61K 31/519; A61K 31/4741; G01N 33/505; A61K 45/06; A61K 39/39

(57) ABSTRACT

Pharmaceutical compositions comprising a compound selected from the group consisting of Compound Nos. 1, 2, 3, 4, 5, 6, 7, 13, 22, 23, 24 and 25, as described in Table 1, and a pharmaceutically acceptable excipient. The pharmaceutical composition of the invention may further comprise an antigen, and/or an adjuvant. Also provided are methods of inhibiting a regulatory T (Treg) cell-mediated immune suppression, or more generally a method for enhancing immune response using a pharmaceutical composition comprising a ligand for human Toll-like receptor (TLR) 8 which activates the MyD88-IRAK4 signalling pathway. The present invention further provides a method of screening for an inhibitor of Treg cells' suppressive activity of host immune response using CD4⁺ Treg cells which express CD25, GITR and FoxP3; secrete IL-10, and are able to suppress the activation of CD4⁺ T cells.

### CROSS REFERENCE TO RELATED APPLICATION

This application claims priority to U.S. Provisional Patent Application No. 61/781,024, filed Mar. 14, 2013. The disclosure thereof is incorporated herein in its entirety by reference.

### FIELD OF THE INVENTION

The present invention relates to immunology, specifically to methods of enhancing a host's immune response, more specifically to methods of reversing the ability of T-reg cells to suppress host immune responses.

### BACKGROUND OF THE INVENTION

Harnessing the immune system to eradicate infectious agents, malignant cells and so on is a promising therapeutic approach. Until recently, however, its application in cancer therapy had met with only sporadic clinical success (Di Lorenzo et al., 2011; Lesterhuis et al., 2011; Rosenberg, 2011). Recent FDA approval of the immunotherapy-based vaccine/drug sipuleucel-T (Provenge®) and ipilimumab (Yervoy®) represents milestones in the field of cancer immunotherapy (Hodi et al., 2010; Kantoff et al., 2010). Furthermore, a phase III clinical trial of gp100 peptide for melanoma also produced highly encouraging clinical results (Schwartzentruber et al., 2011). Nevertheless, the clinical benefits reported for these agents have fallen far short of complete responses and permanent cures. In the case of sipuleucel-T, a survival benefit of only 4.1 months was reported, without objective tumor regression or substantial change in PSA level, while the costs of these infusions was US$93,000. Hence, there remains a critical need to develop more effective and affordable vaccines/drugs for patients with cancer, including metastatic prostate cancer.

Among many factors that may contribute to the relatively low clinical efficacy of peptide-based vaccines and even FDA-approved vaccine therapies (Buonerba et al., 2011), potent negative regulatory mechanisms, including regulatory T (T-reg or Treg) cell-mediated immune suppression in the tumor microenvironment, are a major obstacle to improving therapeutic efficacy of cancer vaccines and drugs (Curiel et al., 2004; Wang et al., 2004; Wang and Wang, 2007; Zou, 2006). For example, preexisting CD4⁺ regulatory T (Treg) cells at tumor sites may potently inhibit antitumor immune response, thus posing major obstacles to effective cancer immunotherapy (Wang et al., 2004; Wang et al., 2005; Wrzesinski and Restifo, 2005). CD4⁺ Treg cell-mediated immune suppression is well documented in animal tumor models and cancer patients (Berendt and North, 1980; Mukherji et al., 1989), and increased proportions of CD4⁺ CD25⁺ Treg cells in the total CD4⁺ T cell populations have been detected in individuals with different types of cancers, including lung, breast and ovarian tumors (Curiel et al., 2004; Woo et al., 2001). Studies by the present inventors further demonstrated the presence of antigen-specific CD4⁺ Treg cells at tumor sites, where they induced antigen-specific and local immune tolerance (Wang et al., 2004; Wang et al., 2005). Hence, overcoming immune suppression may be a key to successful development of more effective cancer vaccines and drugs. Some investigators have attempted to deplete CD4⁺ CD25⁺ Treg cells with an anti-CD25⁺ antibody (Mahnke et al., 2007; Morse et al., 2008; Powell et al., 2008; Rech and Vonderheide, 2009), while others have used cyclophosphamide to deplete Treg cells (Audia et al., 2007; Ghiringhelli et al., 2007). However, anti-CD25 antibodies (Ontak and Daclizumab) and cyclophosphamide are not specific for Treg cell depletion, because CD25 is expressed on both CD4⁺ CD25⁺ Treg and newly activated effector T cells.

Treg cells can be divided into different subsets based on phenotypes, cytokine profiles or suppressive mechanisms. Naturally occurring CD4⁺ CD25⁺ Treg cells representing a small population of CD4⁺ T cells are derived from thymus without antigen stimulation and can suppress immune responses through a cell contact-dependent mechanism (Sakaguchi, 2004; Shevach, 2002). By contrast, antigen-induced Treg cells, such as Tr1 and Th3, are elicited in the peripheral after stimulation with antigens and, in general, suppress immune responses through suppressive cytokines IL-10 and/or TGF-β. (Levings et al., 2002; Shevach, 2002; von Boehmer, 2005). Our previous studies demonstrate that antigen (LAGE1 or ARTC1)-specific CD4⁺ Treg cells suppress immune responses through a cell contact-dependent mechanism shared by naturally occurring CD4⁺ CD25⁺ Treg cells (Wang et al., 2004; Wang et al., 2005). The suppressive function of both naturally occurring CD4⁺ CD25⁺ Treg cells and LAGE1-specific Treg cells could be directly reversed by TLR8 ligands such as Poly-guanosine oligonucleotides (PolyG-OND) in the absence of DCs (Peng et al., 2005). Because CD4⁺ Treg cells are enriched at tumor sites, we hypothesized that different subsets of these Treg cells might be present among tumor-infiltrating lymphocytes (TILs) from cancer patients. Studies to test this prediction identified a novel subset of antigen-specific CD4+ Treg cells, whose immune suppressive function is mediated by soluble factors other than IL-10 or TGF-β (or both).

Although treatment with PolyG-OND resulted in functional reversal of Treg cells (Kiniwa et al., 2007; Peng et al., 2005; Peng et al., 2007), it is not clear whether Poly-G OND could reverse the suppressive function of human and murine Treg cells.

There is a strong need for methods and therapeutic agents that can be used to manipulate the immune suppressive ability of Treg cells.

### SUMMARY OF THE INVENTION

Described herein are the generation and characterization of a new subset of CD4⁺ Treg cells that suppress immune response through IL-10 or TGF-β-independent, soluble factor-mediated mechanisms. In efforts to overcome immune suppression and potential problems associated with depletion strategy, the present inventors developed a screening system using newly identified Treg cells, and identified compounds capable of blocking Treg cell suppressive function. Treatment of Treg cells with different treatment time of those newly identified compounds resulted in a different time window to maintain a nonsuppressive state of Treg cells. Because murine TLR8 is not functional, human TLR8 transgenic mice were generated, and it was shown that treatment with the Treg cell-inhibiting compounds of the present invention enhanced antitumor immunity.

The inhibitors of Treg cells' suppressive activity of host immune response are able to enhance the host's immune responses, and may be used for treating diseases or conditions wherein a patient's immune system is desired to be enhanced, such as cancer or infectious disease.

Compounds, and pharmaceutical compositions comprising the compounds, for inhibiting or enhancing Treg cells' suppressive activity of host immune response, including but not limited to compounds represented by Compounds Nos. 1, 2, 34, 5, 6, 7, 13, 22, 23, 24 and 25 as listed in Table 1 are disclosed.

Accordingly, in one embodiment, the present invention provides a pharmaceutical composition comprising a pharmaceutically effective amount of a compound selected from the group consisting of Compound Nos. 1, 2, 34, 5, 6, 7, 13, 22, 23, 24 and 25, which are described in Table 1, and a pharmaceutically acceptable excipient. The pharmaceutical composition of the invention may further comprise an antigen, which may be a peptide antigen, a protein antigen, a polynucleotide antigen, or a polysaccharide antigen. In one embodiment, the pharmaceutical composition of the present invention may further comprise an adjuvant.

In another embodiment, the present invention provides a method for inhibiting a regulatory T (Treg) cell-mediated immune suppression, or more generally a method for enhancing immune response, in a mammal in need thereof, the method comprising administering to the mammal a pharmaceutically effective amount of a pharmaceutical composition comprising a ligand for human Toll-like receptor (TLR) 8 which activates the MyD88-IRAK4 signaling pathway. The ligand is selected from the group consisting of ssRNA40, ssRNA33, CpG, Poly-G10, resiquimod, loxoribine, flagellin, LPS, and Pam3CSK4; or selected from the group consisting of Compound Nos. 1, 2, 34, 5, 6, 7, 13, 22, 23, 24 and 25.

In one embodiment, the mammal is a human. The mammal may be suffering from a cancer or is at risk of developing cancer. An immunogenic amount of a cancer vaccine comprising a cancer-specific antigen may be further administered to the mammal. In another embodiment, an adjuvant is further administered to the mammal. The adjuvant may be administered with the antigen or is coupled to the antigen.

In another embodiment, an effective amount of a chemotherapeutic agent may also be administered to the mammal.

The mammal may also be afflicted with or is at risk of developing an infectious disease.

The present invention further provides a method of screening for an inhibitor of Treg cells' suppressive activity of host immune response, comprising 1) providing a candidate compound, 2) providing CD4⁺ Treg cells, 3) culturing naïve CD4⁺ T cells with the CD4⁺ Treg cells in the presence or absence of the candidate compound, 4) measure the rate of growth of the naïve CD4⁺ T cells in the presence or absence of the candidate compound, and 5) comparing the growth rate in the presence of the candidate compound to the growth rate in the absence of the candidate compound, wherein a candidate compound in the presence of which the growth rate of the naïve CD4⁺ T cells is higher than in its absence is determined to reverse the inhibition of CD4⁺ Treg cells, and is selected as an inhibitor. The CD4⁺ Treg cells express CD25, GITR and FoxP3; secrete IL-10, and are able to inhibit a host's immune responses. The CD4+ Treg cells may be specific to an antigen. In one embodiment, the screening method of the present invention includes culturing the CD4⁺ T cells in the presence of antigen presenting cells (APCs), such as DCs, which present a specific antigen. The growth rate of the CD4⁺ T cells may be determined by the rate of incorporation of [³H]-thymidine into the CD4⁺ T cells.

Also provided are methods of treating a patient in need thereof (for example a cancer patient, or a patient suffering from an infectious disease) using the compounds or pharmaceutical compositions of the present invention, alone or in combination with other therapeutic agents, such as an antigen preparation or a vaccine.

### BRIEF DESCRIPTION OF THE DRAWINGS

FIG. 1 shows the generation and characterization of tumor-reactive CD4⁺ CD25⁺ T cell lines or clones. (A) FACS analysis of CD4⁺ CD25⁺ T cells among naive CD4⁺ T cells or TIL108 cells from a cancer patient. (B) Analysis of the antigen specificity of T cell clones derived from TIL108. T cell clones were tested against a panel of tumor targets and 293 cells expressing HLA-DR1, DR4 or DR7 molecules. 586mel, 1363mel, 1558mel and 164mel expressed HLA-DR1 molecules, while 108mel and 1359mel were positive for HLA-DR7 and -DR4, respectively. GM-CSF release from T cells was determined by ELISA.

FIG. 2 shows the cytokine profiles and FACS analyses of CD4⁺ T cells. (A) Cytokine profiles of T cell clones. (B) FACS analysis of T cell clones. T cells were stained with phycoerythrin (PE)- or FITC-labeled mAb to CD4, CD25, and GITR molecules. Isotype control antibodies served as negative controls. (C) Determination of Foxp3 expression levels in TIL108 T cell clones by real-time PCR. TIL1363-Th cells served as a control. HPRT served as an internal control.

FIG. 3 shows the functional characterization of the suppressive activity of TIL108 Treg cells. (A) Suppressive activity of TIL108 CD4⁺ Treg cell clones. All TIL108 Treg cell clones suppressed the proliferative response of naïve CD4⁺ T cells, while CD4⁺ effector 1363-Th cells enhanced the proliferative activity of naïve CD4⁺ T cells. (B) A cell-contact mechanism is not required for suppression. Naive CD4⁺ T (responding) cells and Treg cell clones were separated in a transwell system. The suppression of naive T cell proliferation in outer wells was observed even when TIL108 Treg cell clones were cultured in inner wells. (C) Anti-IL-10 and anti-TGF-β antibodies failed to restore naive T cell proliferation. (D) The suppression of naive T cell proliferation was mediated by culture supernatants from TIL108 Treg cells. Ten microliters of culture supernatants from Treg cell clones were sufficient to suppress naive T cell proliferation. (E) Titration of culture supernatants required for the suppression of naive T cell proliferation.

FIG. 4 shows the inhibition of proliferation and IL-2 secretion of CD8⁺ and CD4⁺ effector T cells by TIL108 supernatants. (A) Suppression of proliferation of CD8⁺ effector cells by culture supernatants of TOL108 Treg cells. Supernatants harvested from TIL108 Treg cell clones inhibited the proliferation of CD8⁺ effector cells, while supernatants from either naive CD4⁺ T cells or TIL1359 cells failed to suppress CD8⁺ T cell proliferation. Different amounts of supernatants were used, as indicated. (B) Inhibition of IL-2 secretion of CD4⁺ effector cells by TIL108 supernatants. Supernatants from TIL108 Treg cells treated with OKT3 antibody inhibited IL-2 production by TIL1363 CD4⁺ effector cells. By contrast, supernatants from either untreated TIL108 Treg cells or TIL1558-Th cells treated with OKT3 antibody failed to suppress IL-2 secretion by TIL1363 CD4⁺ effector T cells. (C) IL-2 secretion from TIL1363 CD4⁺ effector cells was better inhibited by TIL108 Treg cells treated with OKT3 antibody than by their supernatants. TIL108 Treg cells were pretreated with OKT3 or control antibody for 12 h, and then washed with T cell assay medium. TIL1558-Th cells served as a control. These T cells were separately mixed with TIL1363 CD4⁺ T cells and the corresponding 1363mel target cells.

FIG. 5 shows the reversal of suppressive activity of TIL108 Treg cell supernatants after the treatment of TIL108 Treg cells. (A) Reversal of the suppressive function of TIL108 Treg cells by Poly-G10 oligonucleotides. (B) Supernatants from Poly-G10-treated TIL108 Treg cells lost the ability to suppress naïve T cell proliferation. Supernatants from the untreated Treg cells served as a control. (C) Knockdown of TLR8, MyD88 and IRAK4 by specific siRNAs in Treg cells blocked the reversibility of Treg cells to suppress naïve T cell proliferation. TLR7 and TLR9 siRNAs served as controls. (D). The suppressive function of TIL108 Treg cells was reversed by synthetic and natural ligands for human TLR 8, but not by ligands for other TLRs. (E). Supernatants of TIL108 Treg cells treated with synthetic and natural ligands for human TLR8, but not ligands for other TLRs, restored naïve T cell proliferation.

FIG. 6 shows the identification of TLR ligands capable of restoring naïve T cell proliferation in the presence of murine Treg cells. A. Naturally occurring CD4⁺ CD25⁺ Treg cells were isolated and purified by FACS sorting after staining with anti-CD4 and anti-CD25 antibodies. The suppressive activity of Treg cells was determined by a functional assay using naïve T cells as responding cells in the presence of different number of CD4⁺ CD25⁺ Treg cells. Naïve T cells (1×10⁵) plus purified. APC (1×10⁴) were mixed with Treg cells in medium containing 0.5 mg of anti-CD3 antibody. After 56 h of culture, [³H]thymidine was added at a final concentration of 1 mCi/well, followed by an additional 16 h. of culture. The incorporation of [³H]thymidine was measured with a liquid scintillation counter. Experiments were performed in triplicate. Treg cells or APC alone did not respond to anti-CD3 antibody. B. Proliferative assays of naïve T cells/APC were conducted in the 1:0.5 ratio of naïve T cells to Treg cells, with or without TLR ligands. Assay conditions were similar to those in panel A. Pam3CSK4 is a ligand for TLR2, poly(I:C) is a ligand for TLR3, LPS is a ligand for TLR4, flagellin is a ligand for TLR5, loxoribine and resiquimod are synthetic ligands for TLR7, poly-G10 is a ligand only for human TLR8, and CpG is a ligand for TLR9. The working concentration for each ligand was used according to manufacturers' instruction and our own titration experiments.

FIG. 7 shows the expression, function and antitumor immunity of human TLR8 in transgenic mice. (A) Expression of human TLR8 gene in CD4-hTLR8 transgenic mice. Total RNA was isolated from each tissue and cell type and used for RT-PCR analysis of hTLR8 expression. (B) Functional evaluation of human TLR8-expressing Treg cells in response to Poly-G3 OND treatment.

FIG. 8 shows that blocking of Treg cell function by Poly-G3 enhances antitumor immunity. CD4-hTLR8 Tg mice were treated with Poly-G3 or Poly-T10 (control) OND (1.0 μg/mouse) on day −2, −1, and subcutaneously injected 4 different types of tumor cells on day 0. Tumor growth was monitored every 2 days.

FIG. 9 shows the two screening strategies for identification of small molecule compounds for blocking Treg cell suppressive function.

FIG. 10 shows the identification of small molecule compounds for the inhibition of Treg cell function. These small molecule compounds contain a quinolone structure. Those with a marked effect on the proliferation of CD4⁺ naïve T cells (CPM>60K, 50% restoration compared to naïve T cells alone) are considered excellent candidates for further drug development. Naïve T cells alone or CD4⁺ naïve cells plus Treg cells, without any compound serve as controls.

FIG. 11 shows the durability of Treg cell functional reversal after treatment with 3 different compounds and treatment times. Treg cells were pretreated with 3 drugs for 1, 3 and 8 days, and cultured in a drug-free medium until use for testing of their suppressive function.

### DESCRIPTION OF THE INVENTION

The potent ability of CD4⁺ regulatory T (Treg) cells to induce self-tolerance by suppressing host immune responses to self-tissues and tumor cells is well recognized, but very little is understood about the suppressive mechanisms used by different subsets of Treg cells. The present inventor discloses herein a novel subset of antigen-specific CD4⁺ Treg cells with a distinctive immunosuppressive mechanism. This novel subset of antigen-specific CD4⁺ Treg cells resemble other Treg cells in their expression of CD25, GITR and FoxP3 markers and their secretion of IL-10, but inhibit immune responses through soluble factors other than IL-10 and TGF-β. It has been found that the suppressive activity can be reversed by treating the Treg cells with ligands for human Toll-like receptor (TLR) 8, which activates the MyD88-IRAK4 pathway, suggesting a new mechanism in which TLR8-MyD88 signaling regulates the soluble molecules responsible for immune suppression.

Accordingly, the present invention provides a method of reversing the immune-suppressive effect by CD4⁺ regulatory T (Treg) cells of host immune responses. The present invention also provides immune-stimulatory combinations and therapeutic and/or prophylactic methods that include administering an immunostimulatory combination of the present invention to a subject. The present invention also provides a method to screen for molecules that inhibit the immune-suppressive effect by CD4⁺ regulatory T (Treg) cells of host immune responses. Methods and compositions of the present invention can provide an increased immune response and improve the efficacy of certain immunological treatments, especially in the prevention or treatment of cancer with vaccines based on cancer-specific antigens.

Application as Medicaments

The Treg cell inhibitor of the present invention is useful as an agent for prevention and/or treatment of various inflammatory diseases, asthma, atopic dermatitis, nettle rash, allergic diseases (allergic bronchopulmonary aspergillosis, allergic eosinophilic gastroenteritis and the like), nephritis, nephropathy, hepatitis, arthritis, chronic rheumatoid arthritis, psoriasis, rhinitis, conjunctivitis, ischemia-reperfusion injury, multiple sclerosis, ulcerative colitis, acute respiratory distress syndrome, shock accompanied by bacterial infection, diabetes mellitus, autoimmune diseases, transplant rejection, immunosuppression, cancer metastasis, acquired immunodeficiency syndrome and the like.

In some embodiments, the pharmaceutical composition of the present invention may further include an antigen. When present, the antigen may be administered in an amount that, in combination with the other components of the combination, is effective to generate an immune response against the antigen. The particular amount of antigen that constitutes an amount effective to generate an immune response can be readily determined by those of ordinary skill in the art.

The antigen may be administered simultaneously or sequentially with any component of the pharmaceutical composition. Thus, the antigen may be administered alone or in a mixture with one or more adjuvants, substances that stimulate a general immune response (including, e.g., an Treg suppressor of the present invention). In some embodiments, an antigen may be administered simultaneously (e.g., in a mixture) with respect to one adjuvant, but sequentially with respect to one or more additional adjuvants.

Sequential co-administration of an antigen and other components of a pharmaceutical composition can include cases in which the antigen and at least one other component of the pharmaceutical composition are administered so that each is present at the treatment site at the same time, even though the antigen and the other component are not administered simultaneously. Sequential co-administration of the antigen and the other components of the immunostimulatory combination also can include cases in which the antigen or at least one of the other components of the pharmaceutical composition is cleared from a treatment site, but at least one cellular effect of the cleared antigen or other component (e.g., cytokine production, activation of a certain cell population, etc.) persists at the treatment site at least until one or more additional components of the pharmaceutical composition are administered to the treatment site.

The antigen can be any material capable of raising a T_{H1} immune response, which may include one or more of, for example, a CD8⁺ T cell response, an NK T cell response, a γ/σ T cell response, or a TH1 antibody response. Suitable antigens include but are not limited to peptides; polypeptides; lipids; glycolipids; polysaccharides; carbohydrates; polynucleotides; prions; live or inactivated bacteria, viruses or fungi; and bacterial, viral, fungal, protozoal, tumor-derived, or organism-derived antigens, toxins or toxoids. Furthermore, it is contemplated that certain currently experimental antigens, especially materials such as recombinant proteins, glycoproteins, and peptides that do not raise a strong immune response, can be used in connection with Treg cell suppressor compound of the invention. Exemplary experimental subunit antigens include those related to viral disease such as adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever. In certain embodiments, the antigen may be a cancer antigen or a tumor antigen. The terms cancer antigen and tumor antigen are used interchangeably and refer to an antigen that is differentially expressed by cancer cells.

A pharmaceutical composition of the invention can be used to therapeutically treat a condition treatable by a cell-mediated immune response. Such a combination can contain at least a therapeutically effective amount of a Treg cell suppressor, and may further include a therapeutically effective amount of an antigen.

The pharmaceutical compositions can be administered as the single therapeutic agent in the treatment regimen, or in combination with another therapeutic agent, such as antivirals, antibiotics, etc.

Because of their ability to enhance immune responses, pharmaceutical compositions of the invention can be particularly useful for treating conditions such as, but not limited to: (a) viral diseases such as, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenza virus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); (b) bacterial diseases such as diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such as chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; (d) neoplastic diseases, such as, intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers (e.g., cancers identified above); and (e) TH2-mediated, atopic, and autoimmune diseases, such as atopic dermatitis or eczema, eosinophilia, asthma, allergy, allergic rhinitis, systemic lupus erythematosus, essential thrombocythaemia, multiple sclerosis, Ommen's syndrome, discoid lupus, alopecia areata, inhibition of keloid formation and other types of scarring, and enhancing would healing, including chronic wounds.

Some embodiments of the pharmaceutical compositions of the invention may be useful as a vaccine adjuvant for use in conjunction with any material that raises either humoral and/or cell mediated immune response, such as live viral, bacterial, or parasitic antigens; inactivated viral, tumor-derived, protozoal, organism-derived, fungal, or bacterial antigens, toxoids, toxins; self-antigens; polysaccharides; proteins; glycoproteins; peptides; cellular vaccines; DNA vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease. Immunostimulatory combinations of the invention may also be particularly helpful in individuals having compromised immune function. For example, it may be used for treating the opportunistic infections and tumors that occur after suppression of cell mediated immunity in, for example, transplant patients, cancer patients and HIV patients.

For the above purposes, the inhibitors of the present invention may be normally administered systemically or locally, usually by oral or parenteral administration, optionally in combination with other drugs. The doses to be administered are determined depending upon, for example, age, body weight, symptom, the desired therapeutic effect, the route of administration, and the duration of the treatment. In the human adult, the doses per person are generally from 1 ng to 1000 mg, by oral administration, once per several days, once three days, once two days, once a day, or up to several times per day, and from 1 ng to 100 mg, by parenteral administration (preferably intravenous administration), once per several days, once three days, once two days, once a day, or up to several times per day, or continuous administration from 1 to 24 hours per day into a vein. As mentioned above, the dosage may be changed due to various conditions or clinical state. Therefore, there are cases in which doses lower than or greater than the ranges specified above may be used. The inhibitors of the present invention may be administered in various forms such as solid forms for oral administration, liquid forms for oral administration, or forms for parenteral administration, for example, injections, drugs for external use, suppositories, eye-drops, inhalations and the like, optionally in combination with other drugs.

In one embodiment, the present invention provides a method of screening for an inhibitor of Treg cells' suppressive activity of host immune response, comprising 1) providing a candidate compound or a test agent, 2) providing CD4⁺ Treg cells that suppress immune response; 3) culturing naïve CD4⁺ T cells with the CD4⁺ Treg cells that suppress immune response, optionally in the presence of antigen presenting cells such as DCs, in the presence or absence of a candidate compound, 3) measuring the growth rate of the CD4⁺ T cells, (e.g. incorporation of [³H]-thymidine) and comparing the rate of growth in the presence of the candidate compound to the rate in the absence of the candidate compound, wherein an increase in the growth rate in the present of the candidate compound indicates that the compound is an inhibitor of Treg cells' suppressive activity of host immune response. In one embodiment, the CD4⁺ Treg cells suppress immune response of the host through an IL-10 or TGF-β-independent soluble factor-mediated mechanism.

Agents or test compounds that exhibit a significant suppression effect, such as at least about a 20% increase in incorporation of [³H]thymidine, as compared to cultures that were not exposed to the agent (e.g., medium alone or another control), are suitable as suppressors of Treg cell activity. The methods described above can be used to screen libraries of compounds, such as those based on combinatorial chemistry or collections of naturally occurring compounds and their derivatives, On example is Mixture Based Positional Scanning Libraries, designed to provide information on the activity of collections of systematically arranged compounds numbering in the thousands to millions. The positional scanning technology has been used successfully to identify novel enzyme inhibitors, receptor agonists and antagonists, antimicrobial, antifungal, and antiviral compounds (Houghten et al., J. Med. Chem. 42:3743-3778, 1999; Pinilla et al., Nat. Med. 9:118-122, 2003). In addition, this technology has been independently validated by a number of research groups. Publications from more than 100 separate studies carried out by approximately 50 research laboratories (Houghten et al., J. Med. Chem. 42:3743-3778, 1999) reflect the broad utility of screening systematically arranged collections of compounds, such as positional scanning libraries.

The following examples are provided for illustrative purpose only and that various modifications or changes in light thereof will be suggested to persons skilled in the art and are to be included within the spirit and purview of this application and scope of the appended claims.

### Examples

### Materials and Methods

T Cell Lines and Clones.

CD4⁺ tumor-infiltrating lymphocytes (TIL108) were cultured from a fresh tumor sample surgically removed from a melanoma patient. All TILs and T cell clones were grown in RPMI 1640 medium containing 10% human AB serum and recombinant IL-2 (300 IU/ml). T cell clones were generated from TIL108 by limiting dilution methods (at 0.3 cell/well) as previously described (Wang et al., 2002). To obtain optimal expansion, we used the OKT3 expansion method as previously described (Wang et al., 2002). TIL1363-Th and TIL1558-Th were CD4⁺ cell clones established from TIL1363 and TIL1558, respectively, and were either identical or similar to previously described clones (Wang et al., 2004). Their designations are intended to reflect the properties that set them apart from other CD4⁺ Treg cells: namely their secretion of Th1 cytokines and their ability to enhance the proliferation of naïve CD4⁺ T cells in response to anti-CD3 antibody. Cytokine release from T cells was determined as previously described (Wang et al., 2004).

FACS Analysis.

Expression of GITR was determined after staining T cells with an anti-GITR antibody (R&D Systems) followed by a secondary goat anti-mouse mAb conjugated to FITC. T cells were maintained in culture medium containing 300 IU/ml of IL-2 for at least 2 weeks before FACS analysis. To determine the expression of CD4, CD25 and GITR, we stained T cells with the respective antibodies (BD Biosciences) conjugated to either PE or FITC. After washing, the cells were analyzed by FACSscan.

Proliferation Assays.

Naïve CD4 T cells (1×10⁵) were cultured with regulatory T cells at different ratios (1:0.2, 1:0.1 and 1:0.05) in anti-CD3 mAb-coated (2 μg/ml) 96-well plates. Alternatively, supernatants from either TIL108 Treg cells or other effector cells (TIL1363-Th and TIL1558-Th) were added to fresh assay medium to make a total volume of 200 μl for proliferation assays. After 56 h of culture, proliferating naïve or effector T cells were labeled with [³H]thymidine at a final concentration of for the last 16 h of incubation. [³H]thymidine incorporation was measured with a liquid scintillation counter. TIL108 Treg cells, in some cases, were treated with Poly-G10, OKT3 antibody or different TLR ligands for 12 h, and then washed with PBS or T cell assay medium. After culturing in fresh medium for 24-36 h, supernatants from these T cells were harvested for measuring their suppressive activity. The following ligands were purchased from Invivogene (San Diego, Calif.): LPS (100 ng/ml), CpG-A (3 μg/ml), CpG-B (3 μg/ml), imiquimod (10 μg/ml), loxoribine (500 μM), poly(I:C) (25 μg/ml), ssRNA40/LyoVec (3 μg/ml), ssRNA33/LyoVec (3 μg/ml), pam3CSK4 (200 ng/ml), flagellin (10 μg/ml), or Poly-G3 (3 μg/ml). Transwell experiments were performed as previously described (Wang et al., 2004).

Real-Time Quantitative PCR Analysis.

Total RNA was extracted from 1×10⁷ T cells with Trizol reagent (Invitrogen, Inc. San Diego, Calif.). A SuperScript II RT kit (Invitrogen, Inc. San Diego, Calif.) was used for reverse transcription. The reverse transcription mixture (20 μl) contained 2 μg of total RNA and was incubated at 42° C. for 1 h. Foxp3 mRNA levels were quantified by real-time PCR using ABI/PRISM7000 sequence detection system (PE Applied Biosystems, Inc. Foster City, Calif.). PCR reactions were performed with primers and an internal fluorescent TaqMan probe specific to Foxp3 or HPRT, purchased from PE Applied Biosystems Inc. (Foster City, Calif.). Expression levels of TLR7, 8 and 9, MyD88 and IRAK4 in each sample were determined by real-time PCR, and normalized with the relative quantity of HPRT, as previously described (Peng et al., 2005).

Construction of Lentivirus-Based siRNAs and Viral Transduction.

Several siRNA sequences (19 nucleotides) for each gene were selected with use of computer-assisted programs. Oligonucleotides containing a siRNA sequence, 8 nucleotide spacers, and a polyT terminator sequence were annealed and then cloned into the HapI and XhoI sites of GFP-expressing pLentilox3.7 vector (Rubinson et al., 2003). siRNAs for IRAK4, MyD88, and TLR7, 8 and 9 and viral transduction were previously described (Peng et al., 2005). Transduction efficiency was analyzed at 3 or 4 days post-transduction, and the cells were sorted into GFP⁺ and GFP⁻ cells with a FACS ARIA sorter. The sorted Treg cells were then used to determine their reversibility by Poly-G10 in functional proliferation assays.

### Results and Discussion

Identification of a CD4⁺ TIL Line with Suppressive Activity

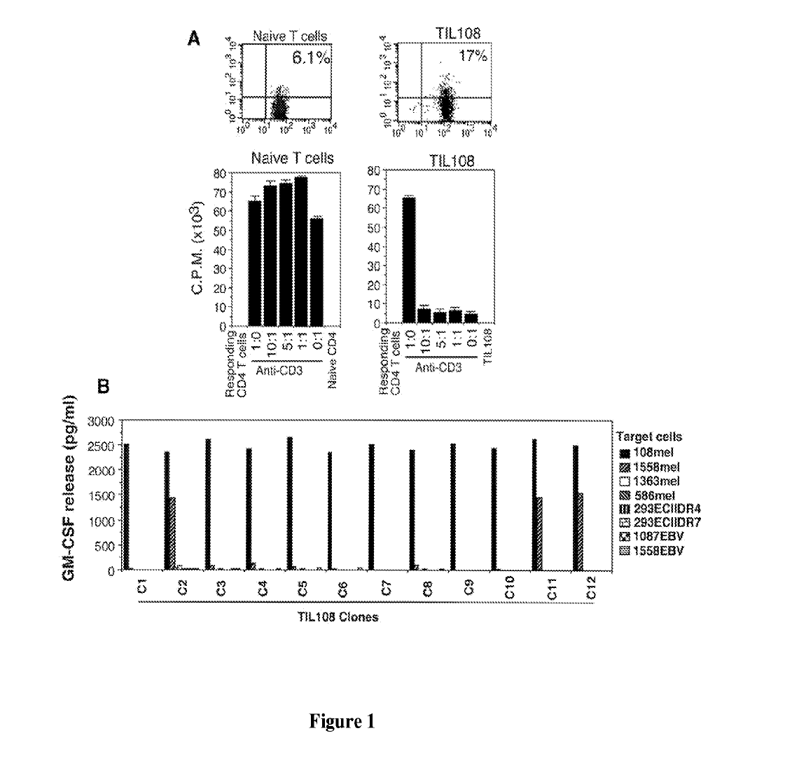

To generate tumor-specific CD4⁺ T cell lines, we first established tumor-infiltrating lymphocytes from fresh tumor samples surgically removed from cancer patients. After depletion of CD8⁺ T cells, the purified CD4⁺ T cell lines were tested against a panel of tumor cell lines. TIL108 was a tumor-reactive CD4⁺ T cell line and was selected for further analysis. FACS analysis revealed that TIL108 contained 17% CD4⁺ CD25⁺ T cells in the total CD4⁺ T cell population, while the normal PBMC-derived CD4⁺ T cell population possessed about 6% CD25⁺ T cells (FIG. 1A). To determine the functional properties of these cells, we showed that the TIL108 cells were capable of suppressing the proliferative response of the purified naïve CD4⁺ T cells to anti-CD3 antibody stimulation (FIG. 1A), while the control naive CD4⁺ T cells failed to inhibit the proliferative response, suggesting that the CD4⁺ TIL108 cell line would be an enriched source of antigen-specific CD4⁺ Treg cell clones.

We next generated CD4⁺ T cell clones from the TIL108 line by a limiting dilution method. Of 35 tumor-reactive CD4⁺ T cell clones, 12 were successfully expanded to obtain a large number of T cells for further analysis. As shown in FIG. 1B, all 12 TIL108 clones specifically recognized. MHC class II-matched 108mel cells, 3 of which recognized allogeneic 1558mel cells, while none of them responded to MHC class II-matched EBV and 293-derived cell lines.

### Phenotypic Analyses of IL-10-Secreting CD4⁺ Treg Cell Clones

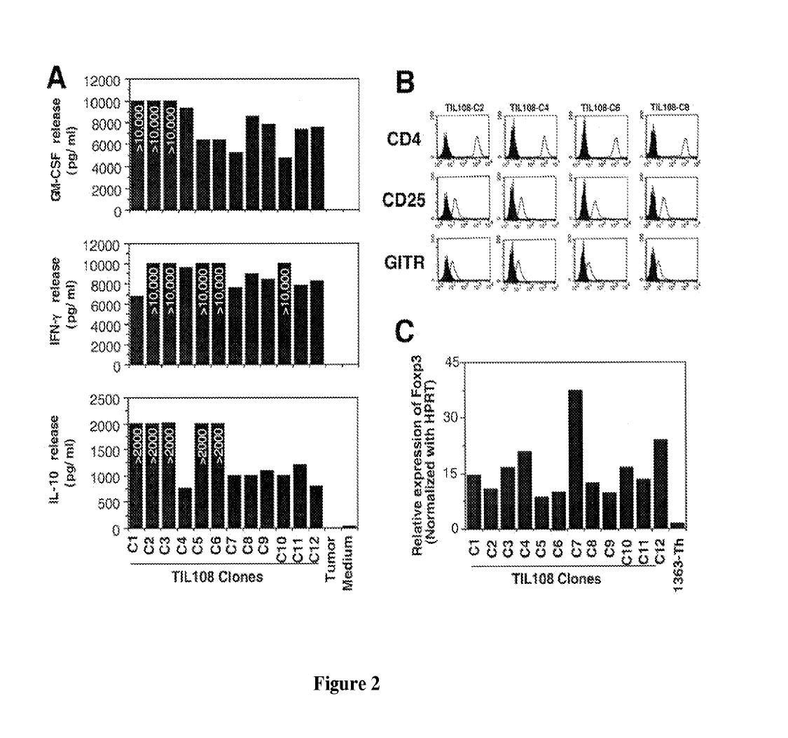

To determine the cytokine profiles for each T cell clone, we found that all 12 T cell clones secreted a large amount of GM-CSF, IFN-γ and IL-10, but little or no other cytokines (FIG. 2A). By FACS analysis, we showed that all IL-10-producing T cell clones expressed the CD4, CD25, and GITR markers. Representative data for 4 clones are shown in FIG. 2B. Real-time PCR analysis revealed that the expression of forkhead transcription factor (Foxp3) in these IL-10-producing CD4⁺ T cell clones was higher than that in CD4⁺ TIL1363-Th cells (FIG. 2C). Taken together, these data indicate that TIL108 T cell clones express markers and cytokines typically associated with CD4⁺ regulatory T cells.

### Soluble Factors Secreted by Treg Cells Mediate Immune Suppression of CD4⁺ T Cells

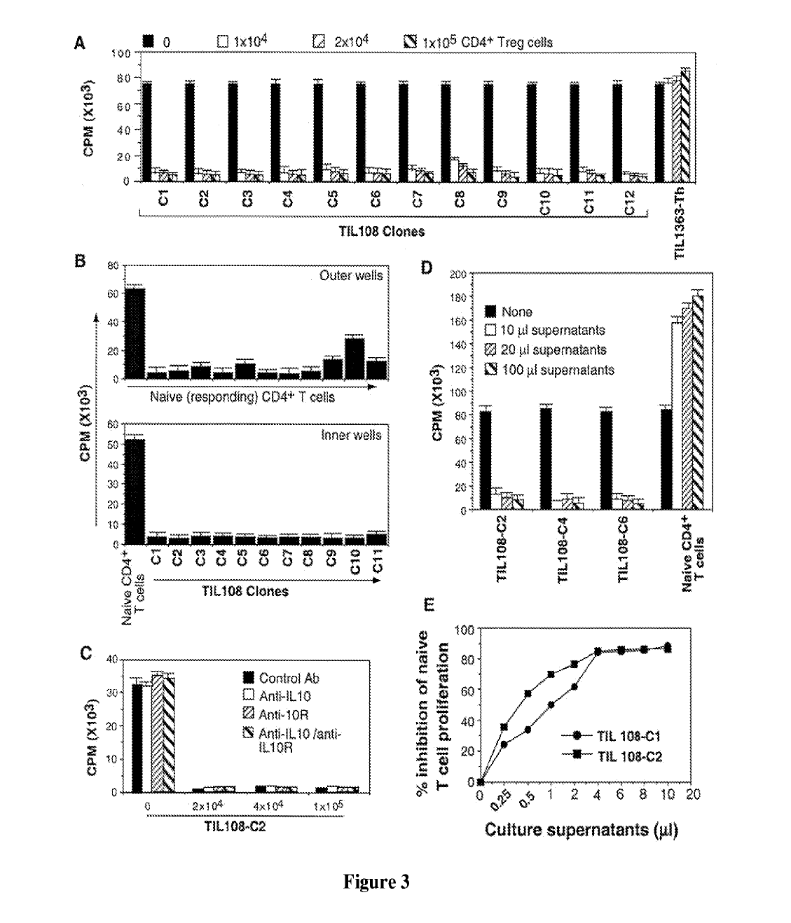

Consistent with the above phenotypic characteristics, all TIL108 Treg cell clones strongly suppressed the anti-CD3-induced proliferation of responding CD4⁺ T cells in a dose-dependent fashion, in contrast to TIL1363-Th effector cells, which enhanced rather than inhibited naïve CD4⁺ T cells proliferation (FIG. 3A). To determine how these TIL108 Treg cells inhibited naïve T cell proliferation, we performed transwell experiments. As shown in FIG. 3B, TIL108 Treg cell clones in the inner wells were still capable of suppressing the proliferation of naïve CD4⁺ T cells in the outer wells, which were not affected by control T cells in the inner wells. Thus, cell-cell contact is not required for the suppressive function of the Treg cell clones.

We next investigated if IL-10 produced in abundance by the Treg cell clones is involved in immune suppression. Inclusion of anti-10, anti-10R or both (10 μg each) in the assay medium failed to restore the proliferative activity of naïve CD4⁺ T cells (FIG. 3C). Similar results were obtained with the addition of a higher amount (30 μg) of antibodies or the use of antibody-coated plates (data not shown). A third antibody, anti-TGF-β, also lacked any effect on the proliferative activity of naïve CD4⁺ T cells (data not shown). These results exclude the participation of IL-10 and TGF-β in the suppression of naïve T cell proliferation mediated by TIL108 Treg cell clones.

To directly test whether the cell culture supernatants were capable of suppressing the proliferation of naïve CD4⁺ T cells, we found that addition of as little as 10 μl of cell culture supernatants from TIL108 Treg cell clones into functional assay medium resulted in over 90% inhibition of the naïve CD4⁺ T cell proliferation, while addition of the same amount of culture supernatants from TIL1363-Th cells or any naïve CD4⁺ T cells enhanced rather than inhibited the proliferation of naïve CD4⁺ T cells (FIG. 3D). Titration experiments showed that suppressive activity increased with increasing amounts of supernatants (FIG. 3E). These results demonstrate that soluble factors secreted by TIL108 Treg cells are directly responsible for their suppressive function. Hence, the TIL108 line/clones may represent a new subset of Treg cells that mediate immune suppression through a mechanism distinct from those employed by naturally occurring CD4⁺ CD25⁺ Treg cells or by Tr1/Th3 cells (Levings et al., 2002; Sakaguchi, 2004; Shevach, 2002). It is likely that additional subsets of Treg cells will be identified as additional clinical samples from cancer patients are evaluated. Besides CD4⁺ Treg cells, other subsets of Treg cells, including CD8⁺ Treg cells, NKT and γδ TCR T cells may also play an important roles in regulating host immune responses in different disease settings (autoimmune diseases and cancer) (Cortesini et al., 2001; Hayday and Tigelaar, 2003; Jiang and Chess, 2004), suggesting the existence of a spectrum of Treg cell subsets with distinctive suppressive mechanisms or phenotypes.

### Inhibition of Effector T Cell Function by TIL108 Treg Cells

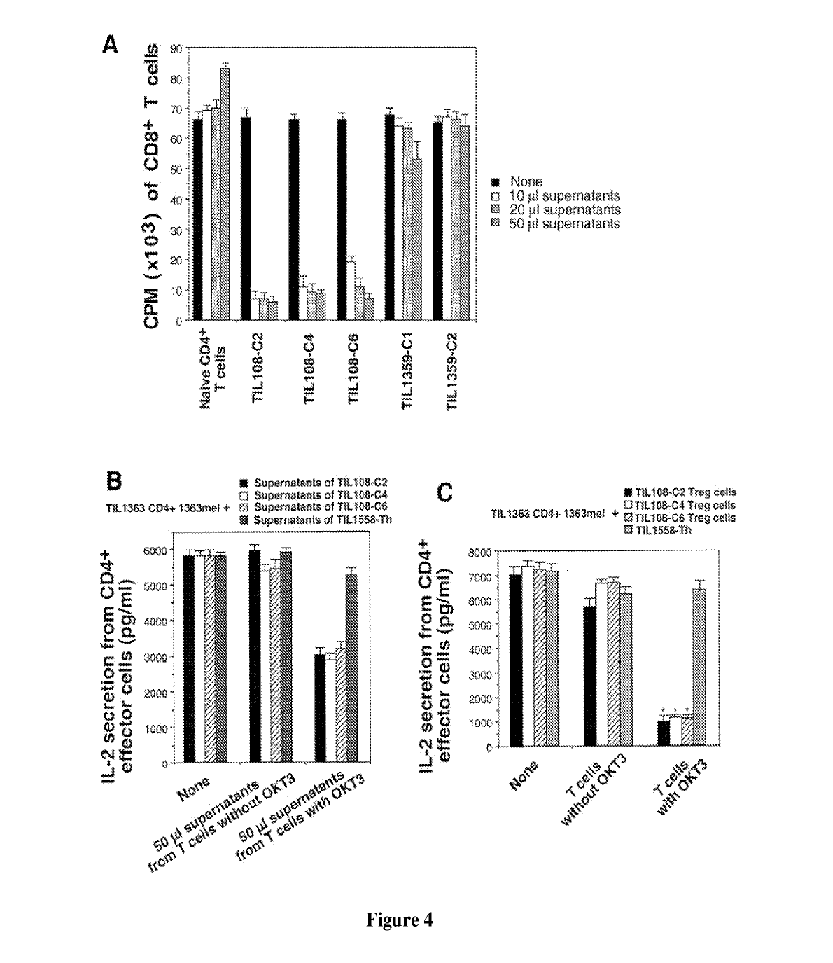

We next tested whether Treg cell supernatants are capable of suppressing the proliferation of antigen-specific CD8⁺ effector cells, As shown in FIG. 4A, the supernatants also strongly inhibited the proliferation of CD8⁺ TIL1359 T cells. To determine whether soluble factors secreted by TIL108 Treg cell clones are capable of inhibiting the ability of TIL1363-Th effector cells to secrete IL-2, we cultured TIL1363-Th cells with 1363mel (stimulator) cells in 150 μl fresh culture medium plus 50 μl of cell supernatants from TIL108 T cell clones, with or without stimulation with an anti-CD3 (OKT3) antibody. The supernatants from cultures of TIL108 Treg cells stimulated with OKT3 inhibited IL-2 secretion by TIL1363-C1 cells, in contrast to supernatants from OKT3-stimulated TIL1558-Th effector cell culture, or from TIL108 Treg cells cultured without OKT3 stimulation, which lacked any activity (FIG. 4B). To further test whether co-culture of TIL108 Treg cells with TIL1363-Th and 1363mel cells results in a better inhibition of IL-2, we found that OKT3-stimulated TIL108 Treg cells showed a stronger inhibition of IL-2 secretion by TIL1363-Th cells in contrast to TIL108 Treg cells without OKT3 stimulation (FIG. 4C). TIL1558-Th cells stimulated with OKT3 antibody did not inhibit IL-2 production by TIL1363-Th cells. These data suggest that activation of TIL108 Treg cells is required for the inhibition of IL-2 production by CD4⁺ Th cells.

### Reversal of the Suppressive Function of TIL108 Treg Cells

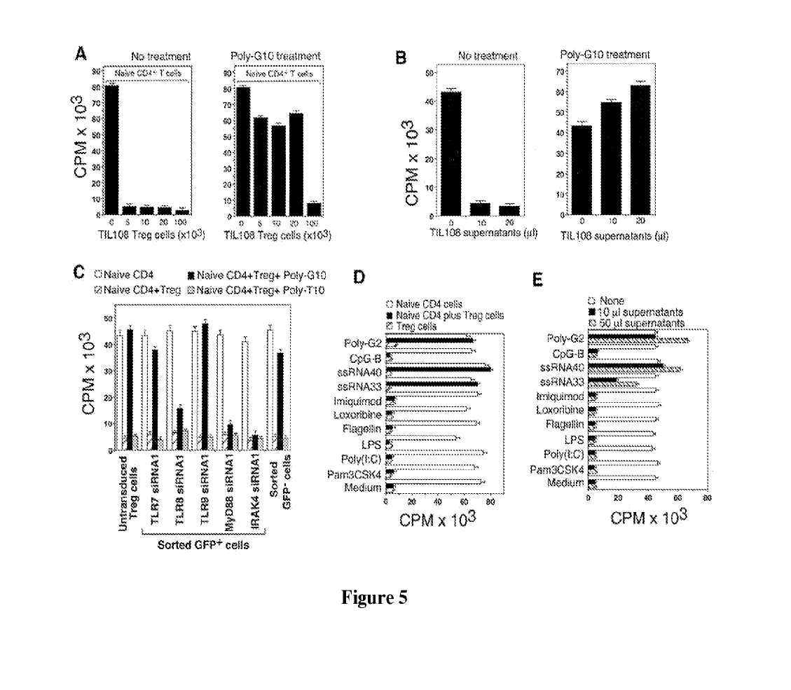

We next tested whether the suppressive function of TIL108 Treg cells can be reversed by Poly-G oligonucleotides. Pretreatment of TIL108 Treg cells with Poly-G10 resulted in reversal of the suppressive function of TIL108 Treg cells and restored the proliferation of naïve CD4⁺ T cells, while untreated TIL108 Treg cells remained suppressive (FIG. 5A). More importantly, supernatants harvested from the Poly-G10-treated TIL108 Treg cells enhanced the proliferation of naïve CD4⁺ T cells, in contrast to supernatants from untreated parental TIL108 Treg cells, which retained their suppressive property (FIG. 5B), suggesting that the suppressive activity of soluble factors secreted by TIL108 Treg cells is directly controlled by a Poly-G-mediated signaling pathway.

We next sought to determine whether the TLR8 signaling pathway is required for the functional reversal of TIL108 Treg cells by Poly-G oligonucleotides. Thus, we knocked down several key molecules of the TLR8 signaling pathway such as TLR8, MyD88 and IRAK4 in TIL108 Treg cells using GFP-expressing lentivirus-based siRNA constructs, which had been demonstrated to inhibit expression of their corresponding genes (Peng et al., 2005). TIL108 Treg cells infected with siRNA virus particles specific for a target gene were sorted into GFP (transduced) and GFP− (untransduced) cells and tested for their ability to respond to Poly-G10 oligonucleotides. As shown in FIG. 5C, specific knockdown of TLR8, MyD88 and IRAK4 by siRNA abolished the ability of Poly-G10 oligonucleotides to reverse suppressive activity of TIL108 Treg cells. By contrast, neither the suppressive activity nor its reversibility was affected when TIL108 Treg cells were transduced with siRNA virus specific for TLR7 and 9. The sorted GFP⁻ TIL108 Treg cells possessed the same reversible suppressive function as the untransduced parental cells (FIG. 5C). Consistent with this result, two natural ligands (ssRNA40 and ssRNA33) for human TLR8 also reversed the suppressive function of TIL108 Treg cells, while ligands for other TLRs failed to restore the proliferation of naïve CD4⁺ T cells in the presence of Treg cells (FIG. 5D). Similar results were obtained with supernatants from Poly-G2 and ssRNA40-treated TIL108 Treg cells, although ssRNA33 ligand was less effective (FIG. 5D).

Because like naturally occurring CD4⁺ CD25⁺ Treg cells as well as LAGE1-specific CD4⁺ Treg cells, the suppressive activity of soluble factors secreted by TIL108 Treg cells is also directly controlled by TLR8 signaling, we speculate that the TLR8-MyD88 signaling pathway specifically controls the function of molecules responsible for the suppressive function of Treg cells, regardless of the specific mechanisms of suppression. Although it is not clear why TLR8, but not other TLR signaling pathways, control the suppressive activity of Treg cells, the expression pattern of TLRs in Treg cells may partially explain this unique property of the TLR8 signaling pathway. Alternatively, TLR8-MyD88-IRAK4 complexes in Treg cells might recruit a unique signaling pathway in the downstream of MyD88-IRAK4 that is required for the control of Treg cell function.

### Modulation of Murine Treg Cell Function Through Distinct TLR Ligands

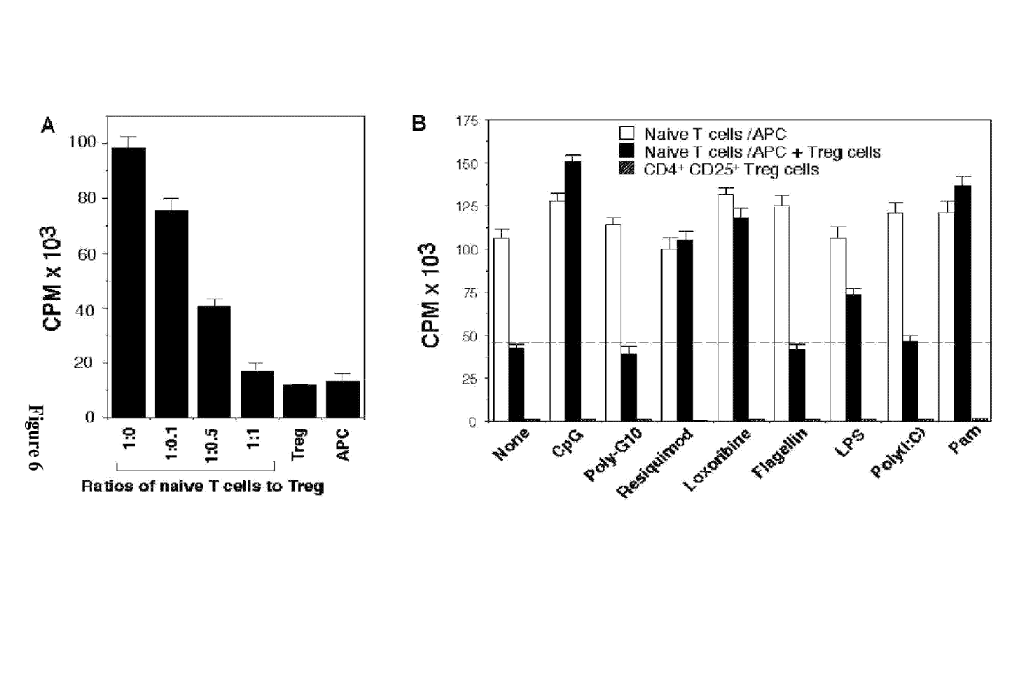

Since TLR8 is not functional in mice (Jurk et al., 2002), it is expected that reversal of Treg cell suppression in mice is different from that in humans. To test whether Poly-G oligonucleotides can trigger murine TLR8 for functional reversal of Treg cells, we isolated and purified murine CD4⁺ CD25⁺ Treg cells and assessed for their ability to respond to TLR ligands. Murine Treg cells were capable of suppressing the proliferation of naïve CD4⁺ T cells, while Treg cells or APCs alone did not proliferate in response to anti-CD3 antibody (FIG. 6A). However, Poly-G oligonucleotides had no effect on reversing the suppressive activity of murine Treg cells (FIG. 6B).

Interestingly, TLR ligands for TLR2, TLR7 and TLR9 could reverse the suppression of naïve T cell proliferation by murine Treg cells (FIG. 6B). LPS, a TLR4 ligand, had a slight effect on reversal of the suppressive function of Treg cells, while ligands for TLR3 and TLR5 lacked any effect on their suppressive function. These data identify the specific TLR pathways that control naïve T cell proliferation in the presence of murine Treg cells, providing rationale to enhance antitumor immunity by shifting the balance between CD4⁺ Treg and T helper cells.

### Development of Human TLR8 Transgenic Models

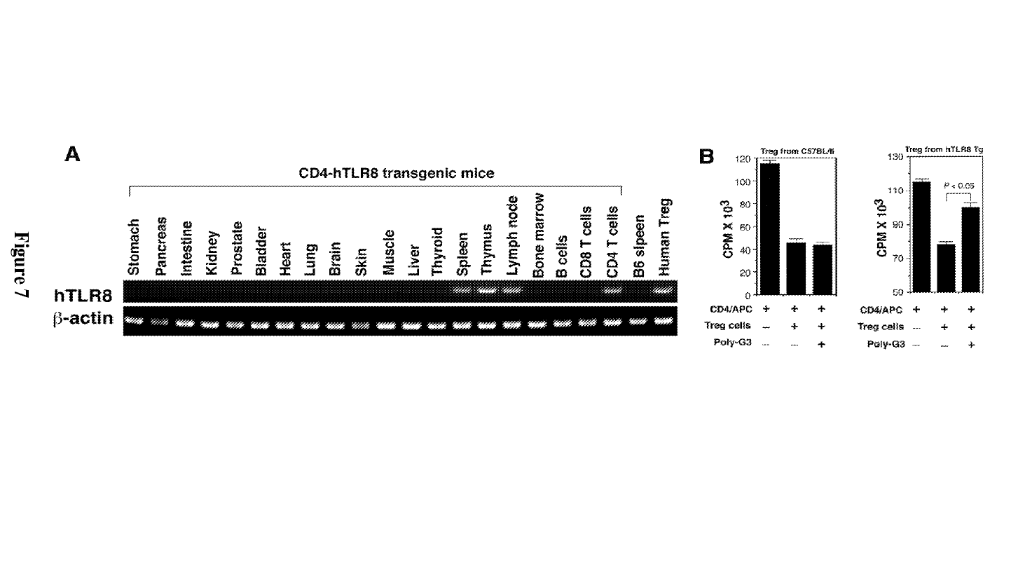

Because murine TLR8 is not functional, we recently generated transgenic (Tg) mice. Human TLR8 is expressed in spleen, thymus, lymph nodes and CD4⁺ T cells, but not in B cells, CD8⁺ T cells or in other tissues analyzed (FIG. 7A). Thus, faithful expression and function of hTLR8 in murine CD4⁺ T cells in CD4-hTLR8 Tg mice will greatly facilitate our ability to manipulate Treg cell function in vivo. Treatment of hTLR8-expressing Treg cells with Poly-G3, but not Poly-T10 (control), reversed their suppressive function (FIG. 7B). These studies suggest that the suppressive function of TLR8-expressing murine Treg cells can be reversed by Poly-G3.

### Enhancing Antitumor Immunity by Blocking Treg Cell Function

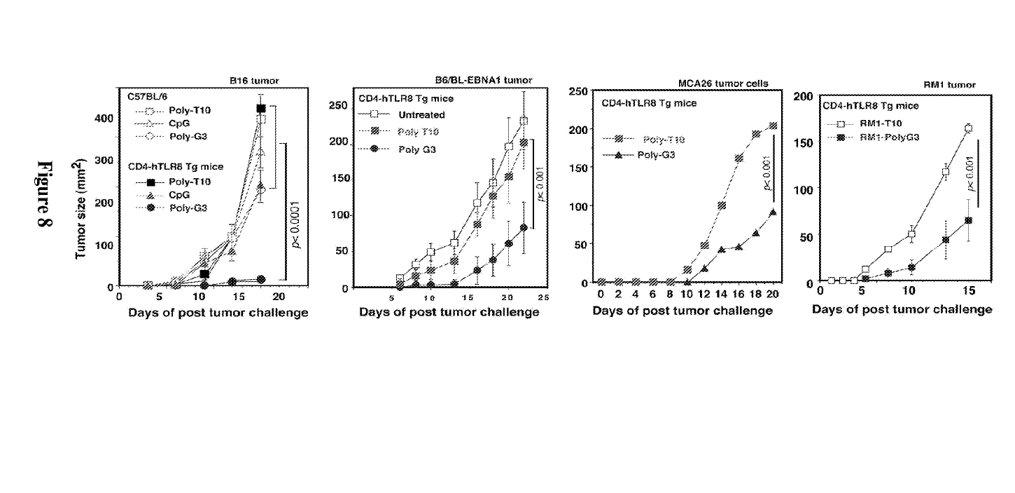

To test whether Poly-G3 treatment enhances antitumor immunity in vivo by blocking Treg cell function, we developed a protective tumor model generated by intravenously injecting Poly-G3, Poly-T10 or CpG oligonucleotides into C57BL/6 wild-type (control) and CD4-hTLR8 Tg mice on days −2 and −1. The treated mice were challenged by subcutaneous injection of B16 tumor cells (1×10⁵ cells/mouse) on day 0, and tumor growth was monitored every 2 days. We found that Poly-G3 treatment enhanced antitumor immunity against B16 cells in CD4-hTLR8 Tg mice, but not in C57BL/6 mice (FIG. 8). Poly-T10 and CpG treatment failed to inhibit tumor growth in both C57BL/6 and CD4-hTLR8 Tg mice, suggesting that TLR8 expression in CD4⁺ T cells is required for Poly-G3-induced antitumor immunity. Importantly, we demonstrated that Poly-G3 treatment inhibited prostate RM1 tumor cell growth in CD4-hTLR8 Tg mice (FIG. 8). Similar results were obtained with several other types of cancer, including lymphoma, MCA28 sarcoma, and RM1 prostate cancer tumor cells.

Identification of New Compounds with the Ability to Reverse the Suppressive Function of Human Treg Cells

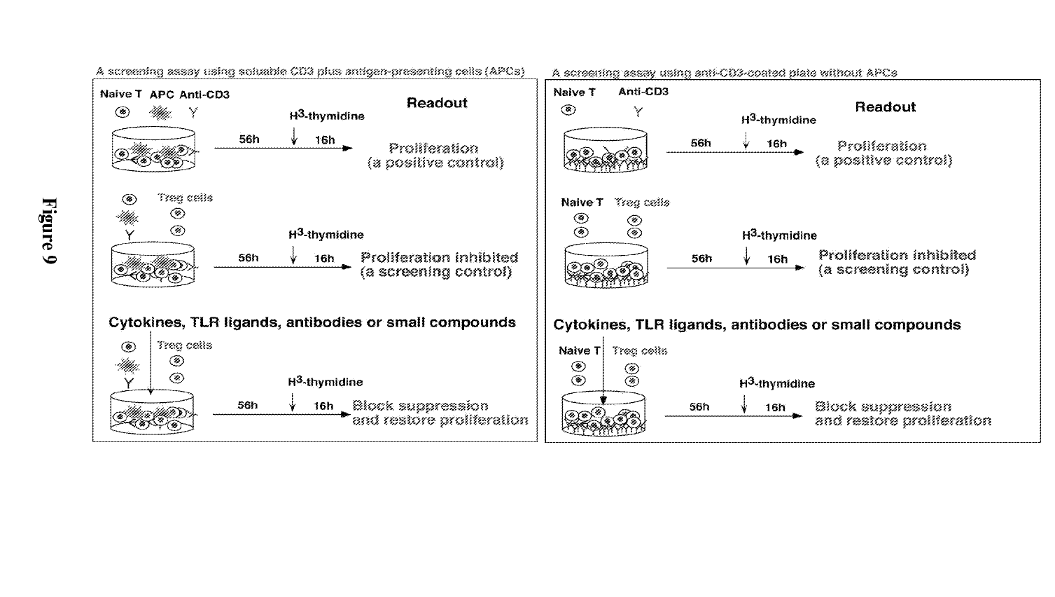

Because our knowledge of the TLR8 signaling pathway is limited at present, an alternative approach will be to screen small molecule compounds. To demonstrate the feasibility of this approach, we have obtained several hundred small molecule compounds from Timtec, Inc., and screened them in experiments similar to those described in FIG. 9.

Specifically, a functional screening assay was carried out using soluable anti-CD3 antibody plus antigen-presenting cells (APCs). Naïve CD4⁺T cells were purified from PBMCs using microbeads (Miltenvi Biotec). DCs were generated from PBMC-derived monocytes in the presence of IL-4 (500 ng/ml) and GM-CSF (800 ng/ml) for 7 days. 1×10⁵ naïve CD4⁺ T cells were cultured with regulatory T cells at different ratios of 1:0.2, 1:0.1 and 1:0.05 in 200 μl of medium containing 2×10⁴ of DCs and anti-CD3 antibody (100 ng/ml) in the absence or presence of various small molecule compounds (1 μM). □ After 56 hours of culture, [³H]thymidine was added at a final concentration of 1 μCi/well, followed by an additional 16 h. of culture. The incorporation of [³H]thymidine was measured with a liquid scintillation counter. All experiments were performed in triplicate. In addition, a screening assay was performed usinge anti-CD3 antibody coated without APCs: 1×10⁵ naïve CD4 T cells were cultured with regulatory T cells at different ratios of 1:0.2, 1:0.1 and 1:0.05 in anti-CD3 mAb-coated (2 μg/ml) 96-well plates in the absence or presence of various small molecule compounds (1 μM) □. After 56 h of culture, [³H]thymidine was added at a final concentration of 1 μCi/well, followed by an additional 16 h of culture. The incorporation of [³H]thymidine was measured with a liquid scintillation counter.

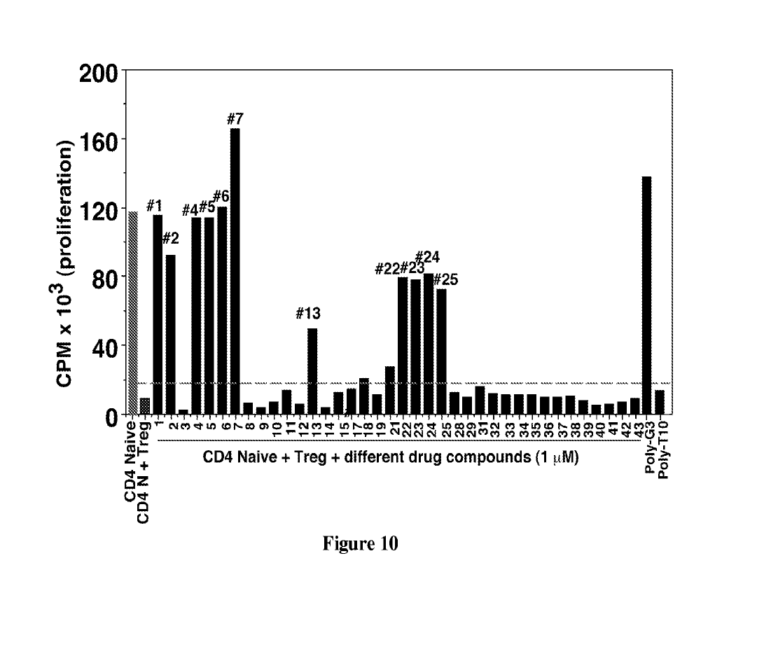

We identified 11 compounds that potently reverse the suppressive function of Treg cells (FIG. 10, and Table 1). These results suggest that small molecule compounds are capable of reversing human Treg cell suppressive function.

### Durability of Drug-Induced Blocking of Treg Cell Suppressive Function is Dependent Upon the Treatment Time

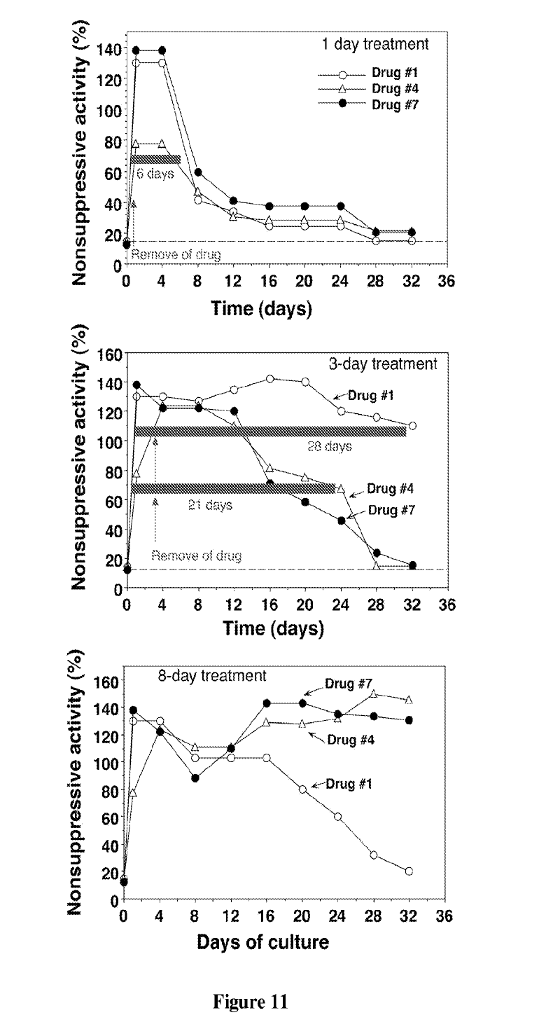

To determine the durability of drug-induced reversal of Treg cell suppressive function, we cultured Treg cells (5×10° each) in the presence of drugs (at a low concentration) for 1, 3 and 5 days, respectively. Treg cells cultured without drugs served as a control. Treg cells were harvested at 1, 3 and 8 days and washed 3 times with PBS to remove the resident drugs. Some of these cells (0.5-1×10⁶) were used in a functional assay, while the remaining T cells were cultured in T cell medium containing IL-2 and used in functional assays every 4 days for 3 weeks. As shown in FIG. 11, treatment of Treg cells with drug compounds #1, #4 and #7 for 1 day and then cultured in the medium without drug after washes until use for testing. Pretreated Treg cells lost suppressive function and remained a unsuppressive condition for 5-6 days. For 3 day treatment of Treg cells with a drug, they remained unsuppressive for 3-4 weeks, and then became suppressive again. Similarly, Treg

TABLE 1
List of Compounds

| ID/Compound No. | Structure | Chemical Name |
|---|---|---|
| ST024751 #1 | 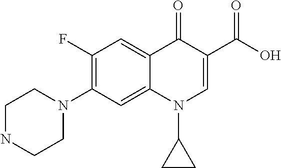 [SMILES: O=C(O)c1cn(C2CC2)c2cc(N3CCNCC3)c(F)cc2c1=O] | 1-Cyclopropyl-6-fluoro-4-oxo-7- (piperazin-1-yl)-1,4- dihydroquinoline-3-carboxylic acid (Common name: Ciprofloxacin) |
| ST020067 #2 | 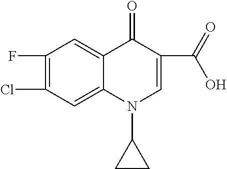 [SMILES: O=C(O)c1cn(C2CC2)c2cc(Cl)c(F)cc2c1=O] | 7-Chloro-1-cyclopropyl-6-fluoro-1,4- dihydro-4-oxoquinoline-3-carboxylic acid |
| ST049451 | 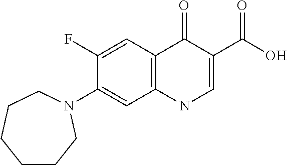 [SMILES: O=C(O)c1c[nH]c2cc(N3CCCCCC3)c(F)cc2c1=O] | 7-(azepan-1-yl)-6-fluoro-4-oxo-1H- quinoline-3-carboxylic Acid |
| ST026704 #4 | 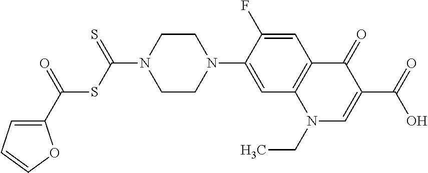 [SMILES: CCn1cc(C(=O)O)c(=O)c2cc(F)c(N3CCN(C(=S)SC(=O)c4ccco4)CC3)cc21] | 1-ethyl-6-fluoro-7-[4-(furan-2- carbonylcarbamothioyl)piperazin-1- yl]-4-oxoquinoline-3-carboxylic acid |
| ST011745 #5 | 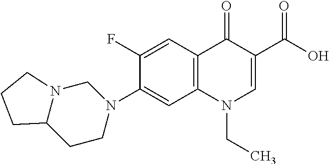 [SMILES: CCn1cc(C(=O)O)c(=O)c2cc(F)c(N3CCC4CCCN4C3)cc21] | 1-Ethyl-6-fluoro-7- (hexahydropyrrolo[1,2-c]pyrimidin- 2(1H)-yl)-4-oxo-1,4-dihydro-3- quinolinecarboxylic acid |
| ST045858 #6 | 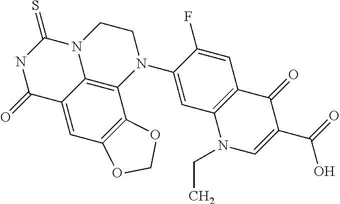 [SMILES: CCn1cc(C(=O)O)c(=O)c2cc(F)c(N3CCn4c(=S)[nH]c(=O)c5cc6c(c3c54)OCO6)cc21] | 7-[4-(1,3-benzodioxole-5- carbonylcarbamothioyl)piperazin-1- yl-1-ethyl-6-fluoro-4-oxoquinoline-3- carboxylic acid |
| ST044514 #7 | 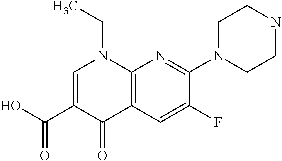 [SMILES: CCn1cc(C(=O)O)c(=O)c2cc(F)c(N3CCNCC3)nc21] | 1-Ethyl-6-fluoro-4-oxo-7-(1- piperazinyI)-1,4-dihydro-1,8- naphthyridine-3-carboxylic acid (Common name: Enoxacin) 6,7-difluoro-4-oxo-1-pentylquinoline- 3-carboxylic acid |
| ST008097 #8 No activity | 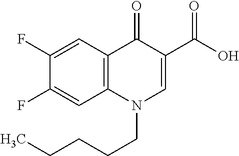 [SMILES: CCCCCn1cc(C(=O)O)c(=O)c2cc(F)c(F)cc21] | 6,7-difluoro-4-oxo-1-pentylquinoline- 3-carboxylic acid |
| ST023837 #9 No activity | 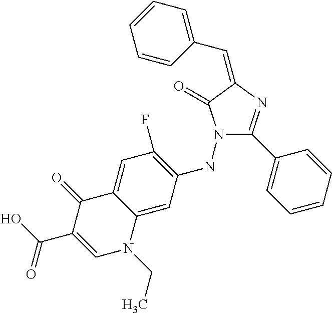 [SMILES: CCn1cc(C(=O)O)c(=O)c2cc(F)c(NN3C(=O)/C(=C\c4ccccc4)N=C3c3ccccc3)cc21] | FLUORESCEIN DIACETATE 5- MALEIMIDE |
| ST020639 #10 No activity | 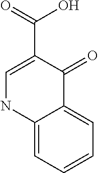 [SMILES: O=C(O)c1c[nH]c2ccccc2c1=O] | 4-oxo-1H-quinoline-3-carboxylic acid |
| ST048777 #11 No activity | 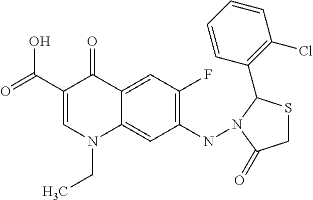 [SMILES: CCn1cc(C(=O)O)c(=O)c2cc(F)c(NN3C(=O)CSC3c3ccccc3Cl)cc21] | {[2-(2-chlorophenyl)-4-oxo(1,3- thiazolidin-3-yl)]amino}-1-ethyl-6- fluoro-4-o xohydroquinoline-3- carboxylic acid |
| ST026709 #12 No activity | 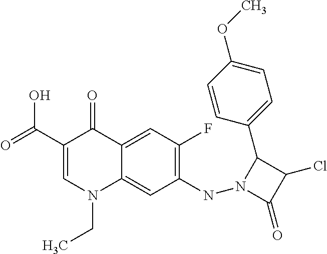 [SMILES: CCn1cc(C(=O)O)c(=O)c2cc(F)c(NN3C(=O)C(Cl)C3c3ccc(OC)cc3)cc21] | 7-{[3-Chloro-2-(4-methoxyphenyl)-4- oxo-1-azetidinyl]amino}-1-ethyl-6- fluoro-4-oxo-1,4-dihydro-3- quinolinecarboxylic acid |
| ST067125 #13 | 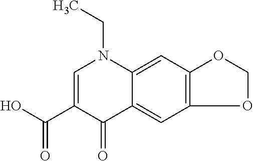 [SMILES: CCn1cc(C(=O)O)c(=O)c2cc3c(cc21)OCO3] | 5-Ethyl-8-oxo-5,8- dihydro[1,3]dioxolo [4,5-g]quinoline- 7-carboxylic acid (Common name: Oxolinic acid) |
| ST067113 #14 No activity | 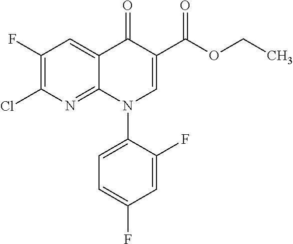 [SMILES: CCOC(=O)c1cn(-c2ccc(F)cc2F)c2nc(Cl)c(F)cc2c1=O] | ethyl 7-chloro-1-(2,4-difluorophenyl)- 6-fluoro-4-oxo-1,8-naphthyridine-3- carboxylate |
| TRE0004030 #15 No activity | 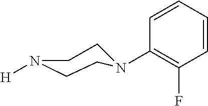 [SMILES: Fc1ccccc1N1CCNCC1] | 1-(2-Fluorophenyl)piperazine |
| TRE0005495 #16 No activity | 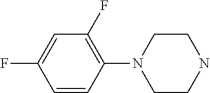 [SMILES: Fc1ccc(N2CCNCC2)c(F)c1] | 1-(2,4-Difluorophenyl)piperazine |
| TRE0005772 #17 No activity | 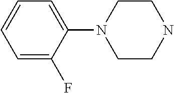 [SMILES: Fc1ccccc1N1CCNCC1] | 1-(2-Fluorophenyl)piperazine |
| TRE0006666 #18 No activity | 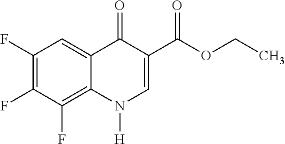 [SMILES: CCOC(=O)c1c[nH]c2c(F)c(F)c(F)cc2c1=O] | Ethyl 6,7,8-trifluoro-1,4-dihydro-4- oxo-3-quinilinecarboxylate |
| TRE0007211 #19 No activity | 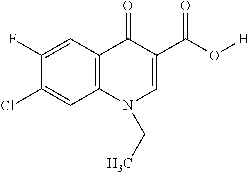 [SMILES: CCn1cc(C(=O)O)c(=O)c2cc(F)c(Cl)cc21] | 7-Chloro-1-ethyl-6-fluoro-4-oxo-1,4- dihydro-3-quinolinecarboxylic acid |
| TRE0008195 #20 No activity | 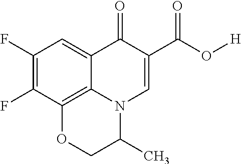 [SMILES: CC1COc2c(F)c(F)cc3c(=O)c(C(=O)O)cn1c23] | 9,10-Difluoro-3-methyl-7-oxo-2,3- dihydro-7H-[1,4]oxazino[2,3,4- ij]quinoline-6-carboxylic acid |
| TRE0065224 #21 No activity | 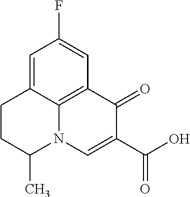 [SMILES: CC1CCc2cc(F)cc3c(=O)c(C(=O)O)cn1c23] | 9-Fluoro-5-methyl-1-oxo-6,7- dihydro-1H,5H-pyrido[3,2,1- ij]quinoline-2-carboxylic acid (Common name: Flumequine) |
| TRE0069035 #22 | 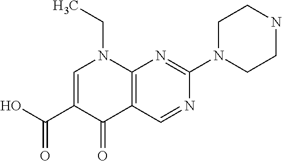 [SMILES: CCn1cc(C(=O)O)c(=O)c2cnc(N3CCNCC3)nc21] | 8-Ethyl-5-oxo-2-(1-piperazinyl)-5,8- dihydropyrido[2,3-d]pyrimidine-6- carboxylic acid (Common name: Pipemidic acid) |
| TRE0073274 #23 | 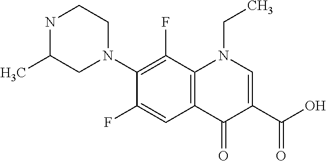 [SMILES: CCn1cc(C(=O)O)c(=O)c2cc(F)c(N3CCNC(C)C3)c(F)c21] | 1-Ethyl-6,8-difluoro-7-(3-methyl-1- piperazinyl)-4-oxo-1,4-dihydro-3- quinolinecarboxylic acid (Common name: Lomefloxacin) |
| TRE0074357 #24 | 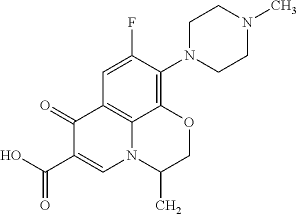 [SMILES: CC1COc2c(N3CCN(C)CC3)c(F)cc3c(=O)c(C(=O)O)cn1c23] | 9-Fluoro-3-methyl-10-(4-methyl-1- piperazinyl)7-oxo-2,3-dihydro-7H- [1,4]oxazino[2,3,4-ij]quinoline-6- carboxylic acid (Common name: Ofloxacin) |
| TRE0074530 #25 | 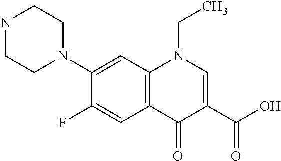 [SMILES: CCn1cc(C(=O)O)c(=O)c2cc(F)c(N3CCNCC3)cc21] | 1-Ethyl-6-fluoro-4-oxo-7-(1- piperazinyl)-1,4-dihydro-3- quinolinecarboxylic acid (Common name: Norfloxacin) |
| TRE0076990 #26 No activity | 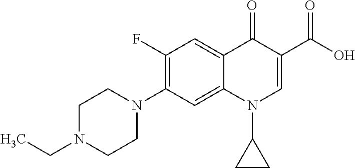 [SMILES: CCN1CCN(c2cc3c(cc2F)c(=O)c(C(=O)O)cn3C2CC2)CC1] | 1-Cyclopropyl-7-(4-ethyl-1- piperazinyl)-6-fluoro-4-oxo-1,4- dihydro-3-quinolinecarboxylic acid (Common name: Enrofloxacin) |
| TRE0080754 #27 No activity | 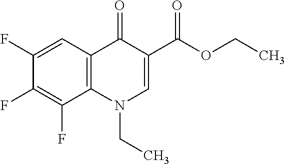 [SMILES: CCOC(=O)c1cn(CC)c2c(F)c(F)c(F)cc2c1=O] | Ethyl 1-ethyl-6,7,8-trifluoro-1,4- dihydro-4-oxo-3- quinolinecarboxylate |
| TRE0080974 #28 No activity | 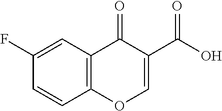 [SMILES: O=C(O)c1coc2ccc(F)cc2c1=O] | 6-Fluoro-4-oxo-4H-chromene-3- carboxylic acid |

cells pretreated for 8 days lost suppressive function and then became suppressive, depending upon different types of compound (FIG. 11). Therefore, the most promising compounds, as determined by the lowest drug concentration required to restore at least 50% of naïve T cell proliferation (i.e. blocking 50% of the suppressive activity of Treg cells) and the ability to maintain a nonsuppressive state for a sufficient time to allow induction of effector T cell immunity, are useful for further experiments. Because the durability of reversal of Treg cell suppressive function is dependent on the treatment time, we could control a time window to induce a maximal antitumor immunity with least risk of autoimmune response. We also expect to identify small molecule compounds that could enhance the suppressive function of Treg cells. Overall, our studies suggest that Treg cell suppressive function could be modulated with different compounds/drugs, which may be important for the treatment of many types of diseases.

All publications, patents and patent applications cited in this patent are hereby incorporated by reference for all purposes. One or more features from any embodiment maybe combined with one or more features of any other embodiment without departing from the scope of the disclosure. The above description is illustrative and is not restrictive. Many variations of the invention will become apparent to those skilled in the art upon review of the disclosure. The scope of the invention should, therefore, be determined not with reference to the above description, but instead should be determined with reference to the claims along with their full scope or equivalents.

### REFERENCES

Audia et al. 2007. Clin. Exp. Immunol. 150:523-530.
Berendt et al. 1980. J. Exp. Med. 151:69-80.
Buonerba et al. 2011. Expert Rev Anticancer Ther 11:25-28.
Cortesini et al. 2001. Immunol. Rev. 182:201-206.
Curiel et al. 2004. Nat. Med. 10:942-949.
Di Lorenzo et al. 2011. Nat Rev Clin Oncol 8:551-561.
Ghiringhelli et al. 2007. Cancer Immunol. Immunother. 56:641-648.
Hayday et al. 2003. Nat Rev Immunol 3:233-242.
Hodi et al. 2010. N. Engl. J. Med. 363:711-723.
Jiang et al. 2004. Adv. Immunol. 83:253-288.
Jurk et al. 2002. Nat Immunol 3:499.
Kantoff et al. 2010. The New England journal of medicine 363:411-422.
Kiniwa et al. 2007. Clinical Cancer Res. 13:6947-6958.
Lesterhuis et al. 2011. Nat Rev Drug Discov 10:591-600.
Levings et al. 2002. Int. Arch. Allergy Immunol. 129:263-276.
Mahnke et al. 2007. Int. J. Cancer 120:2723-2733.
Morse et al. 2008. Blood 112:610-618.
Mukherji et al. 1989. J. Exp. Med. 169:1961-1976.
Peng et al. 2005. Science 309:1380-1384.
Peng et al. 2007. Immunity 27:334-348.
Powell et al. 2008. J. Immunother. 31:189-198.
Rech et al. 2009. Ann. N. Y. Acad. Sci. 1174:99-106.
Rosenberg, 2011. Nat Rev Clin Oncol
Rubinson et al. 2003. Nat. Genet. 33:401-406.
Sakaguchi, S. 2004. Annu. Rev. Immunol. 22:531-562.
Schwartzentruber et al. 2011. The New England journal of medicine 364:2119-2127.
Shevach, E. M. 2002. Nat Rev Immunol 2:389-400.
von Boehmer, H. 2005. Nat Immunol. 6:338-344.
Wang et al. 2004. Immunity 20:107-118.
Wang et al., 2005. J. Immunol. 174:2661-2670.
Wang et al., 2007. Cure. Opin. Immunol. 19:217-223.
Wang et al., 2002. J. Exp. Med. 195:1397-1406.
Woo et al., 2001. Cancer Res. 61:4766-4772.
Wrzesinski et al., Cure. Opin. Immunol. 17:195-201.
Zou, W. 2006. Nat Rev Immunol 6:295-307.

What is claimed is:

1. A pharmaceutical composition comprising a pharmaceutically effective amount of a compound selected from the group consisting of Compound Nos. 1, 2, 3, 4, 5, 6, 7, 13, 22, 23, 24 and 25, and a pharmaceutically acceptable excipient.

2. The pharmaceutical composition of claim 1, further comprising an antigen.

3. The pharmaceutical composition of claim 1, further comprising an adjuvant.

4. The pharmaceutical composition of claim 1, wherein the antigen is a peptide antigen, a protein antigen, a polynucleotide antigen, or a polysaccharide antigen.

5. A method for inhibiting a regulatory T (Treg) cell-mediated immune suppression, or for enhancing immune response, in a mammal in need thereof, the method comprising administering to the mammal a pharmaceutically effective amount of a pharmaceutical composition comprising a ligand for human Toll-like receptor (TLR) 8 which activates the MyD88-IRAK4 signalling pathway.

6. The method of claim 5, wherein the ligand is selected from the group consisting of ssRNA40, ssRNA33, CpG, Poly-G10, resiquimod, loxoribine, flagelllin, LPS, Pam3CSK4.

7. The method of claim 5, wherein the ligand is selected from the group consisting of Compound Nos. 1, 2, 3, 4, 5, 6, 7, 13, 22, 23, 24 and 25.

8. The method of claim 5, wherein the mammal is a human.

9. The method according to claim 5, wherein the mammal has cancer or is at risk of developing cancer, and wherein an immunogenic amount of a cancer vaccine comprising a cancer-specific antigen is further administered to the mammal.

10. The method according to claim 9, wherein an adjuvant is further administered to the mammal.

11. The method according to claim 10, wherein the cancer adjuvant is administered with the antigen or is coupled to the antigen.

12. The method of claim 11, further comprising administering an effective amount of a chemotherapeutic agent.

13. The method of claim 12, further comprising administering an effective amount of a chemotherapeutic agent.

14. The method according to claim 5, wherein the mammal is afflicted with or is at risk of developing an infectious disease.

15. A method of screening for an inhibitor of Treg cells' suppressive activity of host immune response, comprising 1) providing a candidate compound, 2) providing CD4⁺ Treg cells, 3) culturing naïve CD4⁺ T cells with the CD4⁺ Treg cells in the presence or absence of the candidate compound, 4) measure the rate of growth of the naïve CD4⁺ T cells in the presence or absence of the candidate compound, and 5) comparing the growth rate in the presence of the candidate compound to the growth rate in the absence of the candidate compound, wherein a candidate compound in the presence of which the growth rate is higher than in its absence is determined to reverse the inhibition of CD4⁺ Treg cells, and is selected as an inhibitor.

16. The method according to claim 15, wherein the CD4⁺ Treg cells express CD25, GITR and FoxP3; secrete IL-10, and are able to inhibit a host's immune responses.

17. The method according to claim 15, wherein the CD4⁺ Treg cells are specific to an antigen.

18. The method according to claim 17, wherein the CD4⁺ T cells are cultured further in the presence of antigen presenting cells (APCs).

19. The method according to claim 18, wherein the APCs present the antigen.

20. The method according to claim 19, wherein the APCs are dentritic cells.

21. The method according to claim 15, wherein the growth rate is determined by the rate of incorporation of [³H]-thymidine into the CD4⁺ T cells.
